(19) United States
(12) Patent Application Publication
(10) Pub. No.: US 2022/0138456 A1
(43) Pub. Date: May 5, 2022

(54) METHOD AND COMPUTER PROGRAM PRODUCT AND APPARATUS FOR DIAGNOSING TONGUES BASED ON DEEP LEARNING

(71) Applicant: National Dong Hwa University, Shoufeng Township (TW)

(72) Inventors: Shi-Jim YEN, Shoufeng Township (TW); Wen-Chih CHEN, Shoufeng Township (TW); Xian-Dong CHIU, Shoufeng Township (TW); Shi-Cheng YE, Yilan City (TW); Yu-Jin LIN, Taipei City (TW); Chen-Ling LEE, Shoufeng Township (TW)

(73) Assignee: National Dong Hwa University, Shoufeng Township (TW)

(21) Appl. No.: 17/099,961

(22) Filed: Nov. 17, 2020

(30) Foreign Application Priority Data
Oct. 30, 2020 (CN) 202011187504.0

(51) Int. Cl. G06K 9/00; G06K 9/66

(52) U.S. Cl. CPC G06K 9/00288; G06K 9/66

(57) ABSTRACT

The invention introduces a method for diagnosing tongues based on deep learning, performed by processing unit of a tablet computer, including: obtaining a shooting photo through a camera module of the tablet computer; inputting the shooting photo to a convolutional neural network (CNN) to obtain classification results of categories, which are associated with a tongue of the shooting photo; and displaying a screen of a tongue-diagnosis application on a display panel of the tablet computer, where the screen includes the classification results of the categories.

### CROSS-REFERENCE TO RELATED APPLICATIONS

This application claims the benefit of priority to Patent Application No. 202011187504.0, filed in China on Oct. 30, 2020; the entirety of which is incorporated herein by reference for all purposes.

### BACKGROUND

The disclosure generally relates to artificial intelligence and, more particularly, to methods, computer program products and apparatuses for diagnosing tongues based on deep learning.

Tongue diagnosis in Chinese medicine is a method of diagnosing disease and disease patterns by visual inspection of the tongue and its various features. The tongue provides important clues reflecting the conditions of the internal organs. Like other diagnostic methods, tongue diagnosis is based on the “outer reflects the inner” principle of Chinese medicine, which is that external structures often reflect the conditions of the internal structures and can give us important indications of internal disharmony. Conventionally, various image recognition algorithms are used to complete the computer-implemented tongue diagnosis. However, the algorithms can only identify limited tongue characteristics related to color. Thus, it is desirable to have methods, computer program products and apparatuses for diagnosing tongues to identity more tongue characteristics than that are recognized by the image recognition algorithms.

### SUMMARY

In an aspect of the invention, the invention introduces a method for diagnosing tongues based on deep learning, performed by processing unit of a tablet computer, including: obtaining a shooting photo through a camera module of the tablet computer; inputting the shooting photo to a convolutional neural network (CNN) to obtain classification results of different categories, which are associated with a tongue of the shooting photo; and displaying a screen of a tongue-diagnosis application on a display panel of the tablet computer, where the screen includes the classification results of the categories.

In another aspect of the invention, the invention introduces a non-transitory computer program product for diagnosing tongues based on deep learning to include program code when executed by a processing unit of a tablet computer to perform steps of the aforementioned method.

In still another aspect of the invention, the invention introduces an apparatus for diagnosing tongues based on deep learning to include a camera module; a display panel; and a processing unit. The processing unit is arranged operably to obtain a shooting photo through the camera module; input the shooting photo to a CNN to obtain classification results of different categories, which are associated with a tongue of the shooting photo; and display a screen of a tongue-diagnosis application on the display panel, where the screen includes the classification results of the categories.

Both the foregoing general description and the following detailed description are examples and explanatory only, and are not restrictive of the invention as claimed.

### BRIEF DESCRIPTION OF THE DRAWINGS

FIG. 1 is a schematic diagram of three phases for establishing and using the convolutional neural network (CNN) for the tongue diagnosis according to an embodiment of the invention.

FIG. 2 is schematic diagram showing the tongue diagnosis according to an embodiment of the invention.

FIG. 3 shows a screen of a tongue-diagnosis application according to an embodiment of the invention.

FIG. 4 is the hardware architecture of a training apparatus or a tablet computer according to an embodiment of the invention.

FIGS. 5 and 6 are flowcharts illustrating methods of deep learning according to embodiments of the invention.

FIGS. 7 and 8 are flowcharts illustrating methods for diagnosing tongues based on deep learning according to embodiments of the invention.

### DETAILED DESCRIPTION

Reference is made in detail to embodiments of the invention, which are illustrated in the accompanying drawings. The same reference numbers may be used throughout the drawings to refer to the same or like parts, components, or operations.

The present invention will be described with respect to particular embodiments and with reference to certain drawings, but the invention is not limited thereto and is only limited by the claims. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent.” etc.)

In some implementations, a tongue-diagnosis application may use various image recognition algorithms to identity characteristics of tongues in images. Conventionally, such algorithms have better recognition results for features that are highly related to colors, such as “tongue color,” “moss color,” etc. However, such algorithms less-effectively identity the tongue characteristics that are not highly related colors, such as “tongue shape,” “tongue coating,” “saliva,” “tooth-marked tongue,” “red spots,” “black spots,” “cracked tongue,” etc.

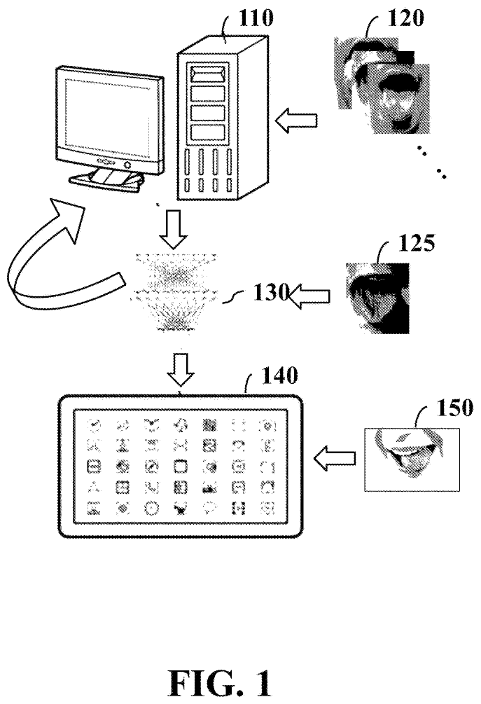

To overcome the drawbacks of the image recognition algorithms, an embodiment of the invention introduces the method for diagnosing tongues based on deep learning, including three phases: training; verification and real-time judgment. Refer to FIG. 1. In the training phase, the training apparatus 110 receives multiple images 120 (also referred to as training images) including a variety of tongues, and tags in each image, where each tag is associated with a specific category. Although the images 120 as shown in FIG. 1 are gray-scale images, there are just examples for illustration. Those artisans may input high-resolution full-color images as a source of training, and the invention should not be limited thereto. The categories may include “tongue color,” “tongue shape,” “moss color,” “tongue coating,” “saliva,” “tooth-marked tongue,” “red spot,” “black spot,” “cracked tongue,” and the like. An engineer may manipulate man machine interface (MMI) of the training apparatus 110 to append tags for different categories to each image 120. For example, for the tongue-color category, an image 120 may be labeled as “light red,” “red,” “light white” or “purple dark.” For the tongue-shape category, an image 120 may be labeled as “normal,” “fat,” “skewed” or “thin.” For the moss-color category, an image 120 may be labeled as “white,” “yellow” or “gray.” For the tongue-coating category, an image 120 may be labeled as “thin moss,” “thick moss,” “greasy moss” or “stripping moss.” For the saliva category, an image 120 may be labeled as “averaged,” “more” or “less.” For the tooth-marked tongue category, an image 120 may be labeled as “yes” or “no.” For the red-spot category, an image 120 may be labeled as “yes” or “no.” For the black-spot category, an image 120 may be labeled as “yes” or “no.” For the cracked-tongue category, an image 120 may be labeled as “yes” or “no.” Each image 120 with tags for different categories may be stored in a non-volatile storage device of the training apparatus 110 in a particular data structure. Subsequently, a processing unit of the training apparatus 110 loads and executes relevant program code to perform deep learning based on the images 120 with their tags for different categories, and the tongue-diagnosis model 130 generated after deep learning will be further verified.

In the verification phase, the training apparatus 110 receives images 125 (also referred to as verification images) including a variety of tongues, and answers in each image, where each answer is associated with a specific category. Subsequently, the verification images 125 are input to the trained tongue-diagnosis model 130 to classify each verification image 125 after proper image pre-processing into resulting items of different categories. The training apparatus 110 compares the answers associated with the verification images 125 with the classification results of the verification images 125 by the tongue-diagnosis model 130 to determine whether the accuracy of the tongue-diagnosis model 130 has passed the examination accordingly. If so, the tongue-diagnosis model 130 is provided to the tablet computer 140; otherwise, the deep learning parameters are adjusted to retrain the tongue-diagnosis model 130.

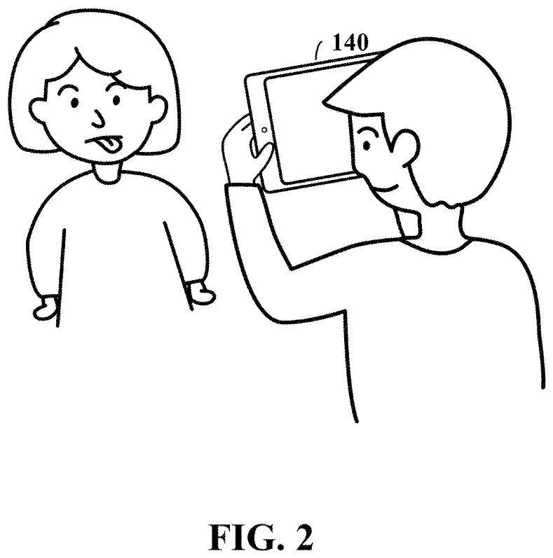

Refer to FIG. 2. In the real-time judgment phase, a doctor picks up the tablet computer 140 to take a picture of a patient. The tong-diagnosis application run on the tablet computer 140 inputs the shooting photo 150 to the tongue-diagnosis model 130 that has been verified to classify the shooting photo 150 after proper image pre-processing into resulting items of different categories. A screen of the tablet computer 140 shows the classification result of each category and the doctor makes more in-depth inquiry and diagnosis for the patient based on the displayed results.

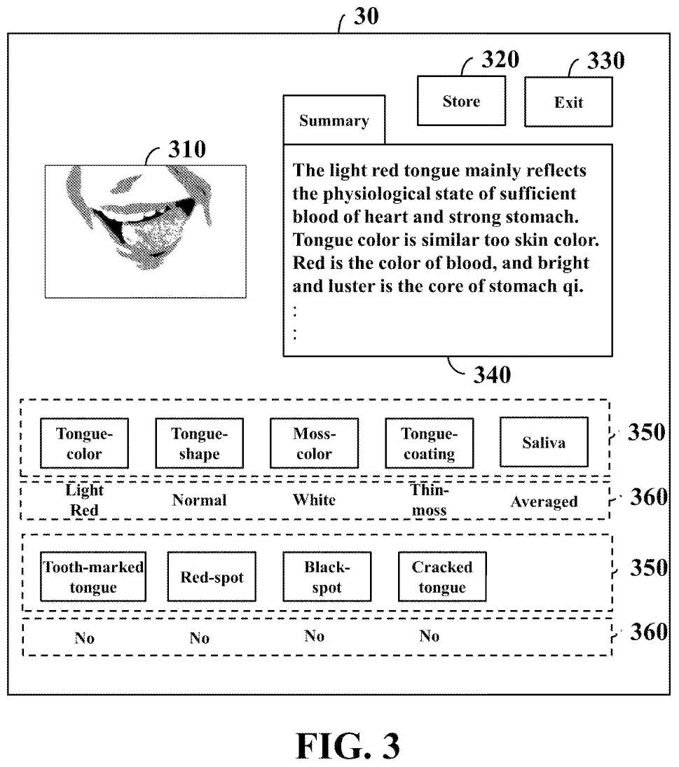

Refer to FIG. 3. The screen 30 of the tongue-diagnosis application includes the preview window 310, the buttons 320 and 330, the result window 340, the category prompts 350 and the classification results 360. The preview window 310 displays the photo of a patient, which is shoot by a camera module of a tablet computer. The category prompts 350 includes, such as “Tongue-color,” “Tongue-shape,” “Moss-color,” “Tongue-coating,” “Saliva,” “Tooth-marked tongue,” “Red-spot,” “Black-spot,” “cracked-tongue,” and the classification results 360 are shown under the category prompts 350. The result window 340 displays summarized textual description for the classification results 360. When the “Store” button 320 is pressed, the tongue-diagnosis application stores the shooting photo 150 and its classification results 360 in a storage device in designated data structure. When the “Exit” button 330 is pressed, the tongue-diagnosis application quits.

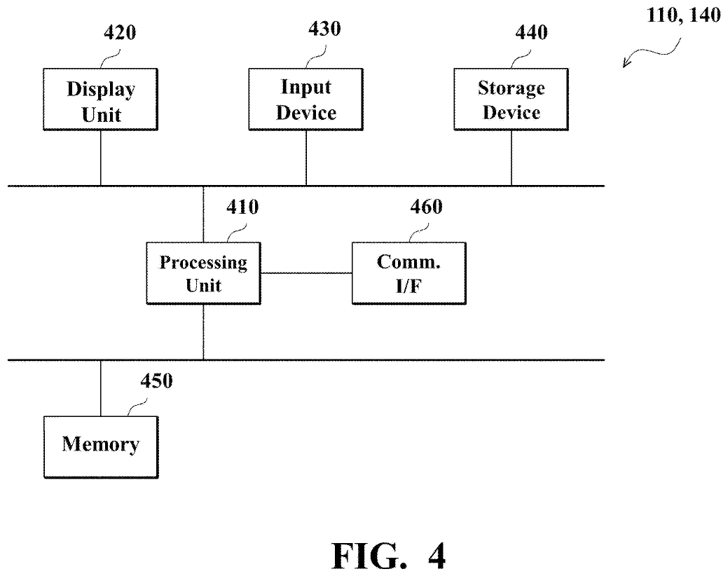

FIG. 4 is the system architecture of a computation apparatus according to an embodiment of the invention. The system architecture may be practiced in any of the training apparatus and the tablet computer 140 to at least include the processing unit 410. The processing unit 410 may be implemented in numerous ways, such as with dedicated hardware, or with general-purpose hardware (e.g., a single processor, multiple processors or graphics processing units capable of parallel computations, or others) that is programmed using program code or software instructions to perform the functions recited herein. The system architecture further includes the memory 450 for storing necessary data in execution, such as images to be analyzed, variables, data tables, data abstracts, the tongue-diagnosis models 130, or others. The system architecture further includes the storage device 440, which may be implemented in a hard disk (HD) drive, a solid state disk (SSD) drives, a flash memory drive, or others, for storing various electronic files, such as the images 120 with their tags for different categories, the tongue-diagnosis models 130, the shooting photo 150 with its classification results for different categories, etc. The communications interface 460 may be included in the system architecture and the processing unit 110 can thereby communicate with the other electronic equipment. The communications interface 460 may be a local area network (LAN) module, a wireless local area network (WLAN) module, a Bluetooth module, a 2G/3G/4G/5G telephony communications module or any combinations thereof. The system architecture may include the input devices 430 to receive user input, such as a keyboard, a mouse, a touch panel, or others. A user (such as a doctor, a patient, an engineer, etc.) may press hard keys on the keyboard to input characters, control a mouse pointer on a display by operating the mouse, or control an executed application with one or more gestures made on the touch panel. The gestures include, but are not limited to, a single-click, a double-click, a single-finger drag, and a multiple finger drag. The display unit 420, such as a Thin Film Transistor Liquid-Crystal Display (TFT-LCD) panel, an Organic Light-Emitting Diode (OLED) panel, or others, may also be included to display input letters, alphanumeric characters and symbols, dragged paths, drawings, or screens provided by an application for the user to view.

In the tablet computer 140, the input device 430 includes a camera module for sensing the R, G and B light strength at a specific focal length, and a digital signal processor (DSP) for generating the shooting photo 150 of a patient according to the sensed values. One surface of the tablet computer 140 may be provided with the display panel for displaying the screen 30 of the tongue-diagnosis application, and the other surface thereof may be provided with the camera module.

In some embodiments for the training phase, the outcome of deep learning (that is, the tongue-diagnosis model 130) may be a convolutional neural network (CNN). The CNN is a simplified artificial neural network (ANN) architecture, which filters out some parameters that are not actually used in image processing, making it uses fewer parameters than that by a deep neural network (DNN) to improve training efficiency. The CNN is composed of convolution layers and pooling layers with associated weights, and a fully connected layer on the top.

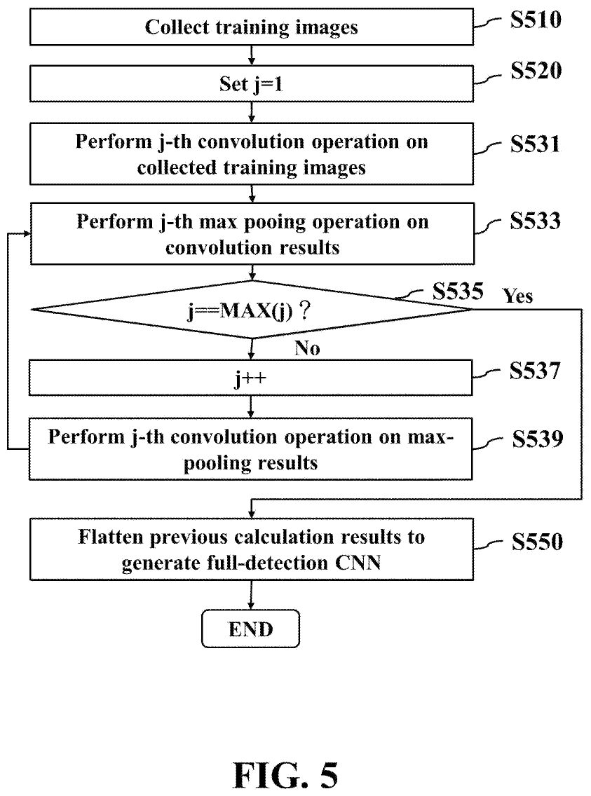

In some embodiments for establishing the tongue-diagnosis models 130, the training images 120 and all the tags of different categories for each training image 120 are input to deep learning algorithms to generate a full-detection CNN for recognizing the shooting photo 150. Refer to FIG. 5 illustrating the deep learning method performed by the processing unit 410 of the training apparatus 110 when loading and executing relevant program code. Detailed steps are described as follows:

Step S510: The training images 120 are collected and each training image is attached with tags of different categories. For example, one training image carries tags of the night categories as {“light white,” “normal,” “white,” “thin moss,” “averaged,” “no,” “yes,” “no,” “yes.”}

Step S520: The variable j is set to 1.

Step S531: The j-th (i.e. first) convolution operation is performed on the collected training image 120 according to their tags of different categories to generate convolution layers and the associated weights.

Step S533: The j-th max pooling operation is performed on the convolution results to generate pooling layers and the associated weights.

Step S535: It is determined whether the variable j equals MAX(j). If so, the process proceeds to step S541; otherwise, the process proceeds to step S537. MAX(j) is a preset constant used to indicate the maximum number of executions of convolution and max pooling operations.

Step S537: The variable j is set to j+1.

Step S539: The j-th convolution operation is performed on the max-pooling results to generate convolution layers and the associated weights.

In other words, steps S533 to S539 form a loop that is executed MAX(j) times.

Step S550: The previous calculation results (such as, the convolution layers, the pooling layers, the associated weights, etc.) are flatten to generate the full-detection CNN. For example, the full-detection CNN is capable of determining the classified item of each of the aforementioned nine categories from one shooting photo.

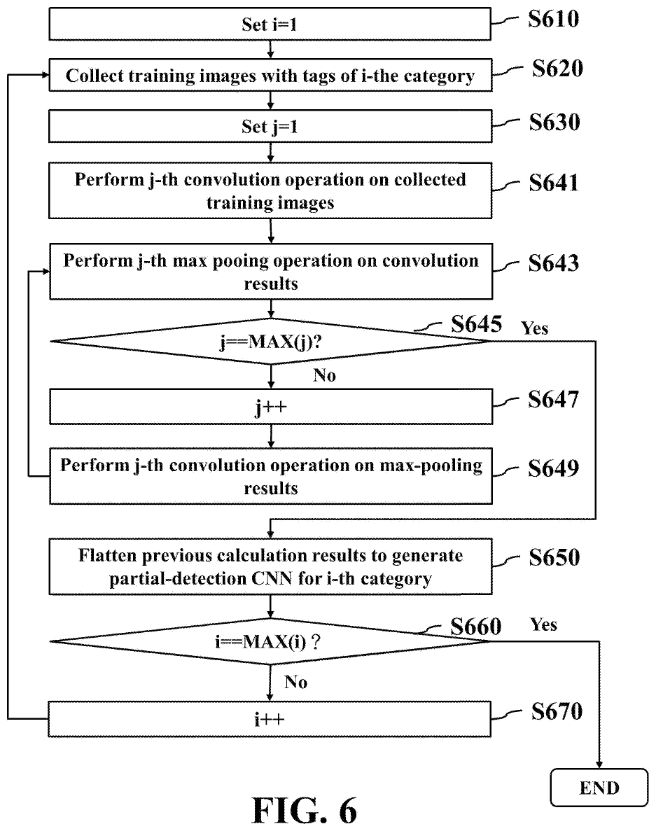

In alternative embodiments for establishing the tongue-diagnosis models 130, multiple partial-detection CNNs are generated and each partial-detection CNN is capable of determining the classified item of one designated category. Refer to FIG. 6 illustrating the deep learning method performed by the processing unit 410 of the training apparatus 110 when loading and executing relevant program code. Detailed steps are described as follows:

Step S610: The variable i is set to 1.

Step S620: The training images 120 are collected and each training image is attached with a tag of the i-th category.

Step S630: The variable j is set to 1.

Step S641: The j-th (i.e. first) convolution operation is performed on the collected training image 120 according to their tags of the i-th category to generate convolution layers and the associated weights.

Step S643: The j-th max pooling operation is performed on the convolution results to generate pooling layers and the associated weights.

Step S645: It is determined whether the variable j equals MAX(j). If so, the process proceeds to step S650; otherwise, the process proceeds to step S647. MAX(j) is a preset constant used to indicate the maximum number of executions of convolution and max pooling operations.

Step S647: The variable j is set to j+1.

Step S649: The j-th convolution operation is performed on the max-pooling results to generate convolution layers and the associated weights.

Step S650: The previous calculation results (such as, the convolution layers, the pooling layers, the associated weights, etc.) are flatten to generate the partial-detection CNN for the i-th category. The partial-detection CNN for the i-th category is capable of determining the classified item of the i-th category from one shooting photo.

Step S660: It is determined whether the variable i equals MAX(i). If so, the process ends; otherwise, the process proceeds to step S670. MAX(i) is a preset constant used to indicate the total number of the categories.

Step S670: The variable i is set to i+1.

In other words, steps S620 to S670 form an outer loop that is executed MAX(i) times and steps S643 to S649 form an inner loop that is executed MAX(j) times.

The processing unit 410 may execute various convolution algorithms known by those artisans to realize steps S531, S539, S641 and S649, execute various max pooling algorithms known by those artisans to realize steps S533 and S643, and execute various flatten algorithms known by those artisans to realize steps S550 and S650, and the detailed algorithms are omitted herein for brevity.

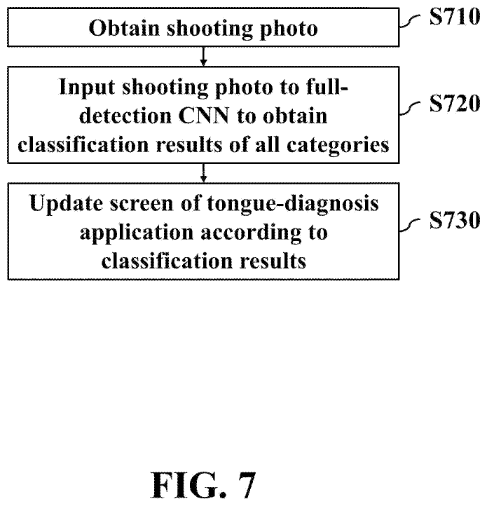

In the real-time judgment phase, if the storage device 440 of the tablet computer 140 stores the full-detection CNN established by the method as shown in FIG. 5, then the processing unit 410 of the tablet computer 140 when loading and executing relevant program code performs the method for diagnosing tongues based on deep learning, as shown in FIG. 7. Detailed steps are described as follows:

Step S710: The shooting photo 150 is obtained.

Step S720: The shooting photo 150 is input to the full-detection CNN to obtain the classification results of all categories. For example, the classification results of the aforementioned nine categories are {“light red,” “normal,” “white,” “thin moss,” “averaged,” “no,” “no,” “no,” “no.”}

Step S730: The classification results 360 of the screen 30 of the tongue-diagnosis application are updated accordingly.

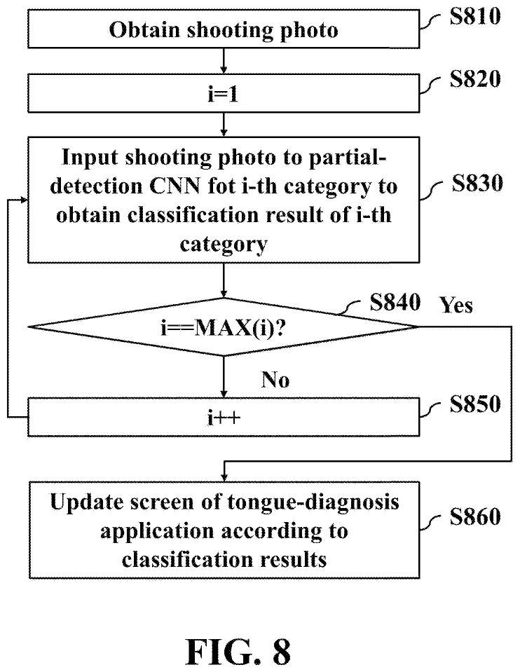

In the real-time judgment phase, if the storage device 440 of the tablet computer 140 stores the partial-detection CNNs established by the method as shown in FIG. 6, then the processing unit 410 of the tablet computer 140 when loading and executing relevant program code performs the method for diagnosing tongues based on deep learning, as shown in FIG. 8. Detailed steps are described as follows:

Step S810: The shooting photo 150 is obtained.

Step S820: The variable i is set to 1.

Step S830: The shooting photo 150 is input to the partial-detection CNN for the i-th category to obtain the classification result of the i-th category.

Step S840: It is determined whether the variable i equals MAX(i). If so, the process proceeds to step S860; otherwise, the process proceeds to step S850. MAX(i) is a preset constant used to indicate the total number of the categories.

Step S850: The variable i is set to i+1.

Step S860: The classification results 360 of the screen 30 of the tongue-diagnosis application are updated accordingly.

Since the numbers of training and verification samples would affect the accuracy and the learning time of deep learning. In some embodiments, for each partial-detection CNN, the ratio of the total numbers of the training images 120, the verification images 125 and the test photo could be set to 17:2:1.

Some or all of the aforementioned embodiments of the method of the invention may be implemented in a computer program, such as program code in a specific programming language, or others. Other types of programs may also be suitable, as previously explained. Since the implementation of the various embodiments of the present invention into a computer program can be achieved by the skilled person using his routine skills, such an implementation will not be discussed for reasons of brevity. The computer program implementing some or more embodiments of the method of the present invention may be stored on a suitable computer-readable data carrier such as a DVD, CD-ROM, USB stick, a hard disk, which may be located in a network server accessible via a network such as the Internet, or any other suitable carrier.

Although the embodiment has been described as having specific elements in FIG. 4, it should be noted that additional elements may be included to achieve better performance without departing from the spirit of the invention. Each element of FIG. 4 is composed of various circuits and arranged to operably perform the aforementioned operations. While the process flows described in FIGS. 5 to 8 include a number of operations that appear to occur in a specific order, it should be apparent that these processes can include more or fewer operations, which can be executed serially or in parallel (e.g., using parallel processors or a multi-threading environment).

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for diagnosing tongues based on deep learning, performed by processing unit of a tablet computer, comprising:
obtaining a shooting photo through a camera module of the tablet computer;
inputting the shooting photo to a convolutional neural network (CNN) to obtain a plurality of classification results of a plurality of categories, which are associated with a tongue of the shooting photo; and
displaying a screen of a tongue-diagnosis application on a display panel of the tablet computer, wherein the screen comprises the classification results of the categories.

2. The method of claim 1, wherein the CNN comprises a plurality of partial-detection CNNs, a total number of the partial-detection CNNs equals a total number of the categories, the method comprising:
inputting the shooting photo to each partial-detection CNN to obtain the classification result of one corresponding category.

3. The method of claim 1, wherein the categories comprise a tongue color, a tongue shape, a moss color, a tongue coating, a saliva, a tooth-marked tongue, a red spot, a black spot and a cracked tongue.

4. The method of claim 1, wherein the screen comprises a preview window to show the shooting photo.

5. A non-transitory computer program product for diagnosing tongues based on deep learning when executed by a processing unit of a tablet computer, the computer program product comprising program code to:
obtain a shooting photo through a camera module of the tablet computer;
input the shooting photo to a convolutional neural network (CNN) to obtain a plurality of classification results of a plurality of categories, which are associated with a tongue of the shooting photo; and
display a screen of a tongue-diagnosis application on a display panel of the tablet computer, wherein the screen comprises the classification results of the categories.

6. The non-transitory computer program product of claim 5, wherein the screen comprises a preview window to show the shooting photo.

7. The non-transitory computer program product of claim 5, wherein the CNN comprises one full-detection CNN, the computer program product comprising program code to:
input the shooting photo to the full-detection CNN to obtain the classification results of the categories.

8. The non-transitory computer program product of claim 5, wherein the CNN comprises a plurality of partial-detection CNNs, a total number of the partial-detection CNNs equals a total number of the categories, the computer program product comprising program code to:
input the shooting photo to each partial-detection CNN to obtain the classification result of one corresponding category.

9. The non-transitory computer program product of claim 8, wherein an establishment of the partial-detection CNN for the i-th category comprises steps of:
performing a convolution operation and a max pooling operation a plurality of times for a plurality of training images according to tags of the i-th category attached with the training images to generate a plurality of convolution layers, a plurality of pooling layers and a plurality of associated weights, wherein i is an integer being greater than 0 and not greater than the total number of the categories;
flattening the convolution layers, the pooling layers and the associated weights to generate a to-be-verified partial-detection CNN for the i-th category;
determining whether the to-be-verified partial-detection CNN for the i-th category is passed an examination according to classification results of the i-th category by inputting a plurality of verification images to the to-be-verified partial-detection CNN; and
generating the partial-detection CNN for the i-th category when the to-be-verified partial-detection CNN for the i-th category has passed the examination.

10. The non-transitory computer program product of claim 9, wherein a ratio of total numbers of the training images and the verification images is 17:2.

11. The non-transitory computer program product of claim 5, wherein the CNN comprises a plurality of convolution layers and a plurality of pooling layers with associated weights, and a fully connected layer on the top.

12. The non-transitory computer program product of claim 5, wherein the categories comprise a tongue color, a tongue shape, a moss color, a tongue coating, a saliva, a tooth-marked tongue, a red spot, a black spot and a cracked tongue.

13. An apparatus for diagnosing tongues based on deep learning, comprising:
a camera module;
a display panel; and
a processing unit, coupled to the camera module and the display panel, arranged operably to obtain a shooting photo through the camera module; input the shooting photo to a convolutional neural network (CNN) to obtain a plurality of classification results of a plurality of categories, which are associated with a tongue of the shooting photo; and display a screen of a tongue-diagnosis application on the display panel, wherein the screen comprises the classification results of the categories.

14. The apparatus of claim 13, wherein the screen comprises a preview window to show the shooting photo.

15. The apparatus of claim 13, wherein the CNN comprises one full-detection CNN, and the processing unit is arranged operably to input the shooting photo to the full-detection CNN to obtain the classification results of the categories.

16. The apparatus of claim 13, wherein the CNN comprises a plurality of partial-detection CNNs, a total number of the partial-detection CNNs equals a total number of the categories, the processing unit is arranged operably to input the shooting photo to each partial-detection CNN to obtain the classification result of one corresponding category.

17. The apparatus of claim 16, wherein an establishment of the partial-detection CNN for the i-th category comprises steps of:
performing a convolution operation and a max pooling operation a plurality of times for a plurality of training images according to tags of the i-th category attached with the training images to generate a plurality of convolution layers, a plurality of pooling layers and a plurality of associated weights, wherein i is an integer being greater than 0 and not greater than the total number of the categories;
flattening the convolution layers, the pooling layers and the associated weights to generate a to-be-verified partial-detection CNN for the i-th category;
determining whether the to-be-verified partial-detection CNN for the i-th category is passed an examination according to classification results of the i-th category by inputting a plurality of verification images to the to-be-verified partial-detection CNN; and
generating the partial-detection CNN for the i-th category when the to-be-verified partial-detection CNN for the i-th category has passed the examination.

18. The apparatus of claim 17, wherein a ratio of total numbers of the training images and the verification images is 17:2.

19. The apparatus of claim 13, wherein the CNN comprises a plurality of convolution layers and a plurality of pooling layers with associated weights, and a fully connected layer on the top.

20. The apparatus of claim 13, wherein the categories comprise a tongue color, a tongue shape, a moss color, a tongue coating, a saliva, a tooth-marked tongue, a red spot, a black spot and a cracked tongue.
